(19) United States
(12) Patent Application Publication
(10) Pub. No.: US 2019/0125725 A1
(43) Pub. Date: May 2, 2019

(54) PHARMACEUTICAL COMPOSITIONS COMPRISING BRIVARACETAM

(71) Applicant: Jubilant Generics Limited, Noida, Uttar Pradesh (IN)

(72) Inventors: Kamal Surendrakumar Mehta, Noida (IN); Dinesh Kumar, Noida (IN); Saurabh Srivastava, Noida (IN)

(21) Appl. No.: 16/094,128

(22) PCT Filed: May 11, 2017
(86) PCT No.: PCT/IB2017/052759

§ 371 Date: Oct. 16, 2018

(30) Foreign Application Priority Data
May 12, 2016 (IN) 201611016571

(51) Int. Cl. A61K 31/4015; A61K 9/20

(52) U.S. Cl. CPC A61K 31/4015; A61K 9/2095; A61K 9/2054; A61K 9/2018; A61K 9/2059; A61K 9/2077

(57) ABSTRACT

The present invention relates to immediate release pharmaceutical compositions comprising Brivaracetam or its pharmaceutically acceptable salts, esters, solvates, derivatives, amides, polymorphs, enantiomers, prodrugs, analogues, active metabolites or mixtures thereof. The present invention also relates to a process for preparing solid oral pharmaceutical composition comprising Brivaracetam. The prior art highlights various technical challenges for the formulation development of Brivaracetam and offers restrictive and complex approach for resolution of technical challenges like use of cyclodextrin. Cyclodextrin free compositions of Brivaracetam prepared as per present invention, exhibited desirable technical attributes.

### FIELD OF THE INVENTION

The present invention relates to pharmaceutical compositions of an anti-epileptic drug or its pharmaceutically acceptable salts, esters, solvates, derivatives, amides, polymorphs, enantiomers, prodrugs, analogues, active metabolites or mixtures thereof. In particular, but without restriction to the particular embodiments hereinafter described in accordance with the best mode of practice, present invention provides solid oral pharmaceutical composition comprising Brivaracetam or its pharmaceutically acceptable salts, esters, solvates, derivatives, amides, polymorphs, enantiomers, prodrugs, analogues, active metabolites or mixtures thereof and process for preparing the same.

### BACKGROUND OF THE INVENTION

Brivaracetam is chemically known as (2S)-2-[(4R)-2-oxo-4-propyltetrahydro-1H-pyrrol-1-yl] butanamide, with the chemical structure as given below:

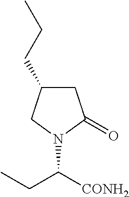

Brivaracetam molecule was disclosed in patent publication WO 01/62726, which describes 2-oxo-1 -pyrrolidine derivatives and methods for their preparation. This patent publication further discloses compound (2S)-2-[(4R)-2-oxo-4-propyl-pyrrolidin-1-yl] butanamide which is known under the international non propriety name as Brivaracetam.

As per Biopharmaceutics Classification System, Brivaracetam is a class I drug (High solubility and permeability). Brivaracetam is reported to exist in two crystalline polymorphic forms designated as polymorph form I and form II. Further, it is also reported that partial conversion from form I to form II may occurs during formulation manufacturing process.

Brivaracetam is commercially-available under the trade name BRIVIACT® in 10, 25, 50, 75, & 100 mg Film Coated Tablets, 10 mg/ml Oral Solution and 50 mg/5 ml (10 mg/ml) Intravenous Injection in the United States for treatment of partial-onset seizures in patients 16 years of age and older with epilepsy.

Inactive ingredients in the BRIVIACT Tablets® consist of Croscarmellose sodium, Lactose monohydrate, Betadex (β-cyclodextrin), Anhydrous lactose, Magnesium stearate. Further, film coating in Briviact Tablets® contains Polyvinyl alcohol, Talc, Polyethylene glycol, Titanium dioxide, Yellow iron oxide, Black iron oxide, and Red iron oxide.

PCT Patent Publications No. WO 2010/086315 and WO 2010/094535 disclose immediate release oral pharmaceutical formulation of 2-oxo-1 -pyrrolidine derivative. These patent publications from UCB Pharma disclose solid oral composition comprising Brivaracetam. These patent publications highlight dissolution, sticking problem (adhesion of tablet formulations to the surfaces of the punches and/or to the dies of the tablet compression machines) associated with Brivaracetam, which is a major challenge during the formulation development. This may causes picking, sticking or other kind of surface irregularities leading to formation of a poor quality finished dosage form. Inventors of these patent publications suggest use of a 0.1% to 60% by weight of cyclodextrin as binding agent. The objective of cyclodextrin as per patent publication is to reduce sticking, improve processability and dissolution profile of tablet formulation of Brivaracetam.

U.S. Pat. Nos. 8,435,564 and 8,563,036 assigned to UCB Pharma discloses prolonged release matrix formulation of Brivaracetam. U.S. Pat. No. 8,460,712 assigned to UCB Pharma and US Patent Publication No. 2013/0039957 assigned to Lupin disclose prolonged release reservoir formulation of Brivaracetam. These patent publications suggest use of either hydrophilic matrix agent or hydrophobic matrix agents or controlled release layer over core to have prolonged drug release such that not more than 40% of drug is released in 1 hour in 900 ml of phosphate buffer, pH 6.4 (Office of Generic Drugs dissolution database) using USP II apparatus (Paddle) at a temperature of 37±0.5° C. and a rotation speed of 50 revolutions per minute (rpm).

Chinese Patent Publication No. CN104800176 assigned to Guangdong Pharmaceutical discloses orally-disintegrating tablet composition of Brivaracetam with high amount of disintegrating agent.

Published literature suggests that formulating Brivaracetam into suitable solid oral dosage forms like tablet is challenging from the formulation development perspective due to sticky nature of API, dissolution issues, compressibility issues, polymorph conversion, manufacturing process selection, control of process and degradation impurities (including chiral impurities).

Hence, there is an unmet need in the art to develop a simple, reproducible, and cost-effective manufacturing process for pharmaceutical composition of Brivaracetam and its derivatives which also offers desired pharmaceutical technical attributes.

It is an object of the present invention to prepare a pharmaceutical composition comprising Brivaracetam or its pharmaceutically acceptable salts, derivatives, solvates, esters, amides, enantiomers, polymorphs or mixtures thereof, wherein the composition is substantially free of cyclodextrin, free of surface irregularities like sticking to a punch or die, free from conversion to other polymorphic form and have desired dissolution profile.

The present inventors have developed an alternate dosage form of Brivaracetam which offer desirable formulation characteristics like compressibility and dissolution. Further, the process employed in the manufacture of dosage form of Brivaracetam is consistent and therefore feasible for industrial production.

### OBJECTS AND SUMMARY OF THE INVENTION

It is a principal object of the present invention to provide a pharmaceutical composition comprising Brivaracetam or its pharmaceutically acceptable salts or derivatives thereof with one or more pharmaceutically acceptable excipient.

It is another object of the present invention to provide a pharmaceutical composition comprising Brivaracetam or its pharmaceutically acceptable salts or derivatives thereof wherein the composition is substantially free of cyclodextrin and optionally free of binder and/or disintegrant.

It is another object of the present invention to provide a pharmaceutical composition comprising Brivaracetam or its pharmaceutically acceptable salts or derivatives thereof and one or more pharmaceutically acceptable excipients, wherein the composition is substantially free of cyclodextrin and free from surface irregularities due to sticking to a punch or die.

It is another object of the present invention to provide a pharmaceutical composition comprising Brivaracetam or its pharmaceutically acceptable salts or derivatives thereof and one or more pharmaceutically acceptable excipients, wherein the composition is substantially free of cyclodextrin and undesired polymorphic forms.

It is another object of the present invention to provide a process for the preparation of a pharmaceutical composition comprising Brivaracetam or its pharmaceutically acceptable salts or derivatives thereof.

It is another object of the present invention to provide a solid oral pharmaceutical composition comprising Brivaracetam or its pharmaceutically acceptable salts or derivatives thereof comprising at least one or more pharmaceutically acceptable excipients like diluent, glidant, surfactant, wetting agent, lubricant, solubilizer, stabilizer, sweetener, flavoring agent, coloring agent and the like.

The following embodiments further describe the objects of the present invention in accordance with the best mode of practice, however, disclosed invention is not restricted to the particular embodiments hereinafter described.

In accordance with a preferred embodiment of the present invention, there is provided pharmaceutical composition comprising Brivaracetam or its pharmaceutically acceptable salts or derivatives thereof wherein the composition is substantially free of cyclodextrin.

In accordance with another embodiment of the present invention, there is provided a pharmaceutical composition comprising Brivaracetam or its pharmaceutically acceptable salts or derivatives thereof wherein composition is substantially free of binder.

In accordance with yet another embodiment of the present invention, there is provided a pharmaceutical composition comprising Brivaracetam or its pharmaceutically acceptable salts and one or more pharmaceutically acceptable excipients, wherein the composition is substantially free of disintegrant.

In accordance with yet another embodiment of the present invention, there is provided a pharmaceutical composition comprising Brivaracetam or its pharmaceutically acceptable salts or derivatives thereof and one or more pharmaceutically acceptable excipient, wherein Brivaracetam is present in an amount of more than 5% by weight based on the total weight of the composition. In particular, the amount of Brivaracetam may vary from about 5% to about 80% by weight, based on the total weight of the composition.

In accordance with still another embodiment of the present invention, there is provided Immediate Release pharmaceutical composition comprising Brivaracetam or its pharmaceutically acceptable salts or derivatives thereof wherein the composition releases more than 85% drug in 45 minutes in 900 ml of phosphate buffer, pH 6.4 (Office of Generic Drugs dissolution database) using USP II apparatus (Paddle) at a temperature of 37±0.5° C. and a rotation speed of 50 revolutions per minute (rpm).

In accordance with still another embodiment of the present invention, there is provided a pharmaceutical composition comprising Brivaracetam or its pharmaceutically acceptable salts or derivatives thereof prepared by wet granulation, dry granulation, dry mixing, direct compression or extrusion-spheronization process.

In accordance with still another embodiment of the present invention, there is provided a process for the preparation of a pharmaceutical composition comprising Brivaracetam or its pharmaceutically acceptable salts or derivatives thereof comprising the steps of: (a) homogeneously mixing or blending therapeutically active compound and one or more pharmaceutically acceptable an excipient (b) compressing the resultant dry homogeneous mixture under sufficient high pressure to form a tablet.

In accordance with still another embodiment of the present invention, there is provided a process for the preparation of a pharmaceutical composition comprising

Brivaracetam or its pharmaceutically acceptable salts or derivatives thereof comprising the steps of a) Sifting the accurately weighed quantities of active agent and one or more pharmaceutically acceptable excipient(s) through a suitable sieve followed by mixing; b) Granulating the mixture of step a) with an suitable granulation liquid (an aqueous or non-aqueous solvent with optional binder); c) Drying the granulated mass, optionally milling of the dried granules and mixing; d) lubricating the sifted blend of step c); e) Compressing the lubricated granules into tablets or filling in capsules.

In accordance with still another embodiment of the present invention, there is provided a process for the preparation of a pharmaceutical composition comprising Brivaracetam or its pharmaceutically acceptable salts or derivatives thereof comprising the steps of: (a) blending a mixture of Brivaracetam and at least one pharmaceutically acceptable excipient; (b) optionally compacting the blended material using roller compactor; (c) optionally milling the compacted material; (d) blend from step (a) or milled material from step (c) was lubricated with a suitable lubricant; (e) lubricated blend or granules from (d) was compressed into tablets with a suitable tooling or filled in capsules.

In accordance with still another embodiment of the present invention, there is provided a pharmaceutical composition comprising Brivaracetam or its pharmaceutically acceptable salts or derivatives thereof and pharmaceutically acceptable excipients, wherein sticking tendency of drug is overcome by using controlled atmospheric condition [temperature of from about 27° C. to about 35° C. and a relative humidity of less than 40%].

In accordance with still another embodiment of the present invention, there is provided a pharmaceutical composition comprising Brivaracetam or its pharmaceutically acceptable salts or derivatives thereof, wherein the compression force used in the manufacturing of composition ranges between about 20 to 400 N/mm².

In accordance with still another embodiment of the present invention, there is provided a pharmaceutical composition comprising Brivaracetam or its pharmaceutically acceptable salts or derivatives thereof, wherein Brivaracetam has a particle size distribution such that D₉₀ is less than 200 μm, D₅₀ is less than about 100 μm and D₁₀ is less than about 50 μm.

In accordance with still another embodiment of the present invention, there is provided a solid oral dosage form comprising a pharmacologically effective amount of Brivaracetam or its pharmaceutically acceptable salts or derivatives thereof wherein, the composition exhibits desired technical attributes like free from polymorphic conversion, desired dissolution, free from surface irregularities such as picking and sticking to punches or die.

In accordance with another embodiment of the present invention, there is provided a pharmaceutical composition comprising Brivaracetam or its pharmaceutically acceptable salts or derivatives thereof and one or more pharmaceutically acceptable excipients, wherein the said composition exhibits hardness of at least 4 Kilopascals (kP) when measured using Monsanto hardness tester.

In accordance with still another embodiment of the present invention, there is provided a pharmaceutical composition comprising Brivaracetam or its pharmaceutically acceptable salts or derivatives thereof, wherein the composition exhibits at least 85% dissolution within 45 minutes when measured in 900 ml of pH 6.4 phosphate buffer solution using USP apparatus type II, at 50 rpm and at 37° C.

In accordance with still another embodiment of the present invention, there is provided a pharmaceutical composition comprising Brivaracetam or its pharmaceutically acceptable salts or derivatives thereof, wherein the composition is substantially free from other polymorphic forms when measured using powder X-ray diffraction method performed on a Bruker D8 Advance powder X-ray diffractometer using Cu κ_{α} radiation (1.546 Å); 2θ angles are recorded with an experimental error of ±0.2°.

In yet another embodiment of the present invention there is provided use of a pharmaceutical composition comprising Brivaracetam or its pharmaceutically acceptable salts or derivatives thereof in the manufacture of a medicament for treating partial-onset seizures, epilepsy, neuropathic pain, essential tremors, Parkinson's disease, dyskinesia, migraine, tremor, essential tremor, bipolar disorders, chronic pain, affective disorders, motor impairment, fibromyalgia, post-herpetic neuralgia, bipolar disorder, schizoaffective disorders, attention disorders, and anxiety disorders.

### BRIEF DESCRIPTION OF THE DRAWINGS

The above and other objects and features of the present invention will become apparent from the following description of the invention, when taken in conjunction with the accompanying drawings, in which:

FIG. 1 shows X-ray powder diffraction pattern of Brivaracetam API (Active Pharmaceutical Ingredient).

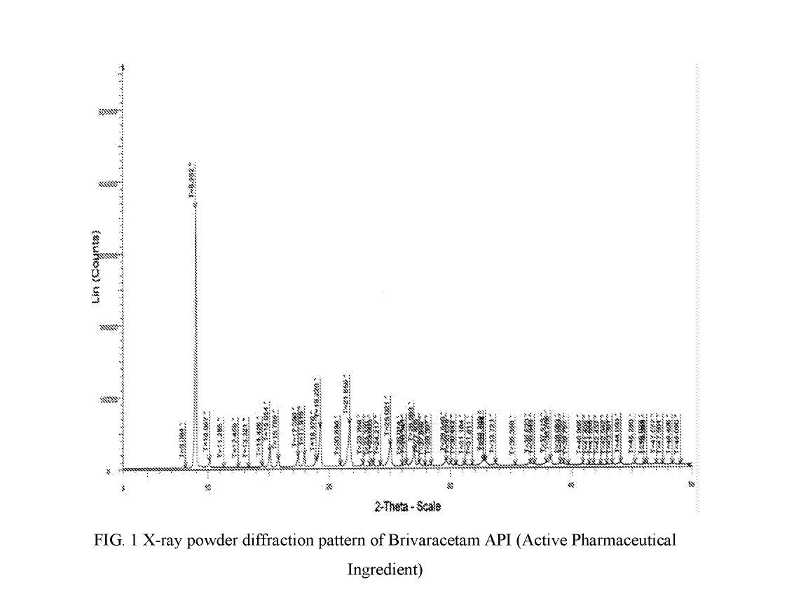

FIG. 2 shows the X-ray powder diffraction pattern of Placebo.

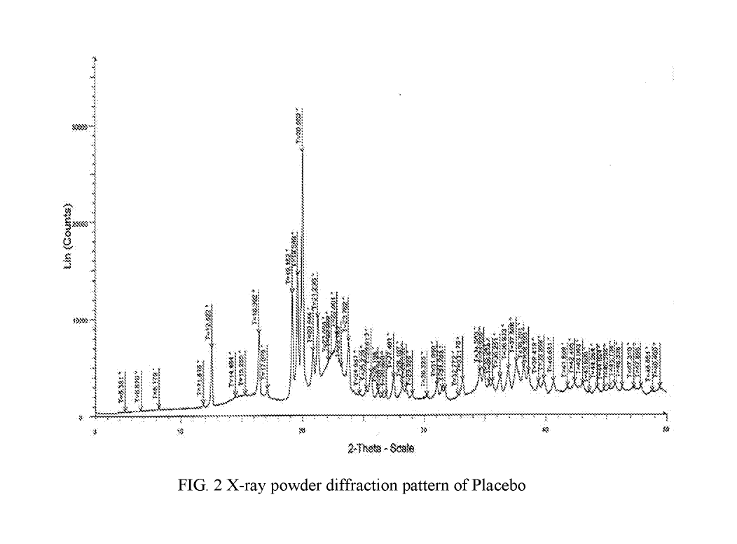

FIG. 3 shows the X-ray powder diffraction pattern of Briveracetam Tablet prepared as per the invention.

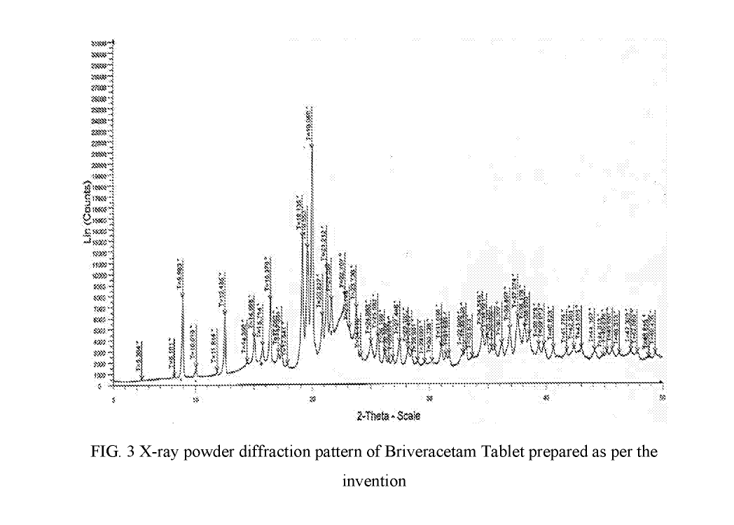

### DETAILED DESCRIPTION OF THE INVENTION

The present invention can be more readily understood by reading the following detailed description of the invention and study of the included examples.

As used herein, the term “composition”, as in pharmaceutical composition, is intended to encompass a drug product comprising an anticonvulsant drug, preferably Brivaracetam or its pharmaceutically acceptable salts or derivatives thereof, and other inert ingredient(s) (pharmaceutically acceptable excipients). Such pharmaceutical compositions are synonymous with “formulation” and “dosage form”. Pharmaceutical composition of the invention include, but is not limited to, granules, tablets (single layered tablets, multilayered tablets, bio adhesive tablets, caplets, matrix tablets, tablet within a tablet, mucoadhesive tablets), capsules (immediate) (hard and soft or liquid filled soft gelatin capsules), pills, troches, sachets, powders, microcapsules, mini-tablets, tablets in capsules and the like. Preferably, the pharmaceutical composition refers to tablets and capsules. More preferably, the pharmaceutical composition refers to immediate release oral tablets, which may be uncoated or film coated.

As used herein, the term “Brivaracetam” is used in broad sense to include not only “Brivaracetam” per se but also its pharmaceutically acceptable pharmaceutically acceptable salts, pharmaceutically acceptable esters, pharmaceutically acceptable solvates, pharmaceutically acceptable derivatives, pharmaceutically acceptable amides, pharmaceutically acceptable polymorphs, pharmaceutically acceptable enantiomers, pharmaceutically acceptable prodrugs, pharmaceutically acceptable analogues, pharmaceutically acceptable active metabolites and also its various crystalline and amorphous forms. The term “Brivaracetam” used in this specification means in substantially pure form, i.e. at least about 95% pure.

The term “excipient” means a pharmacologically inactive component such as a diluent, binder, disintegrant, glidant, surfactant, wetting agent, lubricant, solubilizer, stabilizer sweetener, flavoring agent, coloring agent and the like. The excipients that are useful in preparing a pharmaceutical composition are generally safe, non-toxic and are acceptable for veterinary as well as human pharmaceutical use. Reference to an excipient includes both one and more than one such excipient. Co-processed excipients are also covered under the scope of present invention. Further, excipient may be in the form of powders or in the form of dispersion. Combination of excipients performing the same function may also be used to achieve desired formulation characteristics.

“Substantially free” as used herein refers to the pharmaceutical composition of Brivaracetam, which comprises less than 0.1% w/w cyclodextrin and/or binder and/or disintegrant. According to a particularly preferred embodiment, the pharmaceutical composition is substantially free of cyclodextrin and/or binder and/or disintegrant. Cyclodextrin may be selected from the group consisting of alpha cyclodextrin, beta cyclodextrin, hydroxypropyl beta cyclodextrin, methyl beta cyclodextrin, sulfobutyl beta cyclodextrin, gamma cyclodextrin, hydroxypropyl gamma cyclodextrin or any other which is known in the art.

“Substantially free of other polymorphic forms” as used herein may also refer to the pharmaceutical composition of Brivaracetam, which is free from conversion to other polymorphic forms during formulation development or stability studies

As used in this specification, the singular forms “a”, “an”, and “the” include plural references unless the context clearly dictates otherwise. Thus, for example, a reference to “a process” includes one or more process, and/or steps of the type described herein and/or which will become apparent to those persons skilled in the art upon reading this disclosure and so forth.

As used herein, the term “immediate release” means any type of release of the active ingredient, Brivaracetam, from the composition of the present invention resulting in in-vitro release over a short period of time, i.e., (less than forty five minutes) sufficient to provide therapeutically effective plasma levels over similarly short time interval.

Unless otherwise stated the weight percentages expressed herein are based on the final weight of the composition or formulation.

The pharmaceutical compositions of present invention comprise about 1 to about 500 mg of Brivaracetam, preferably about 2.5 to about 200 mg of Brivaracetam. The pharmaceutical composition comprises Brivaracetam in the range of more than about 5% by weight based on the total weight of the composition. In particular, the amount of Brivaracetam may vary from about 5% to about 80% by weight, based on the total weight of the composition.

In another embodiment the pharmaceutical composition of the present invention includes particle size of Brivaracetam or its pharmaceutically acceptable salts or derivatives thereof having a particle size distribution such that D₉₀ is less than about 200 μm, D₅₀ is less than about 100 μm and D₁₀ is less than about 50 μm. The particle size of Brivaracetam can be measured by suitable techniques such as Laser light scattering (e.g. Malvern Light Scattering), Coulter counter microscopy and any other technique known in the art.

In another embodiment, the present invention includes a solid oral pharmaceutical composition comprising more than about 5% by weight of Brivaracetam based on the total weight of the composition wherein, the composition is substantially free of other polymorphic forms as evidenced by presence of angles of refraction 2 theta (θ), of 8.9 when measured using powder X-ray diffraction method performed on a Bruker D8 Advance powder X-ray diffractometer using Cu κ_{α} radiation (1.546 Å); 2θ angles are recorded with an experimental error of ±0.2°.

In another embodiment, the present invention includes a pharmaceutical composition comprising Brivaracetam or its pharmaceutically acceptable salts or derivatives thereof and one or more pharmaceutically acceptable excipients, wherein the said composition is substantially free of cyclodextrin.

In another embodiment of the invention, there is provided a solid oral pharmaceutical composition comprising Brivaracetam and at least one or more pharmaceutically acceptable excipients like diluent, glidant, surfactant, wetting agent, lubricant, solubilizer, stabilizer, sweetener, flavoring agent, coloring agent and the like.

In another embodiment of the invention, there is provided a pharmaceutical composition comprising Brivaracetam wherein the composition is substantially free of binder and/or disintegrant.

In another embodiment of the invention, the solid oral pharmaceutical composition comprising Brivaracetam is prepared by wet and/or dry process or any other process which is known in the art. The processes include, but are not limited to, wet granulation, dry granulation, dry mixing, direct compression, solid dispersion, melt extrusion, spray drying, precipitation, traditional melt cool method, melt agglomeration, co-precipitation, co-evaporation, solvent evaporation such as vacuum drying, hot plate drying, slow evaporation at low temperature, rotary evaporation, spray drying, freeze drying, spin drying, super critical fluid drying and/or extrusion-spheronization. Other formulation techniques are also contemplated within the scope of the present invention.

Solvents used during preparation of present invention include, but are not limited to, water, dipolar aprotic solvent, polyethylene glycol, polyethylene glycol ether, polyethylene glycol derivative of a mono- or di-glyceride, buffers, esters such as ethyl acetate; ketones such as acetone; alcohols such as methanol, ethanol, isopropanol, butanol; dichloromethane, chloroform, dimethyl acetamide (DMA), dimethyl sulfoxide (DMSO), ether, diethyl ether or any combination thereof.

In another embodiment of the invention, wet granulation can be performed using Rapid mixer granulator, Fluid bed granulator, Planetary mixer and the like; dry mixing can be performed using V-blender or key blender; and dry granulation can be performed using roller compactor or slugging techniques or by any other method known in the art.

In another embodiment of the invention, there is provided a process for the preparation of a pharmaceutical composition comprising Brivaracetam comprising the steps of: (a) homogeneously mixing therapeutically active compound and one or more pharmaceutically acceptable an excipient (b) compressing the resultant dry homogeneous mixture under sufficient high pressure to form a tablet of sufficient abrasion-resistance and solidity for pharmaceutical end uses.

In another embodiment of the invention, there is provided a process for the preparation of a pharmaceutical composition comprising Brivaracetam or its pharmaceutically acceptable salts or derivatives thereof comprising the steps of a) Sifting accurately weighed quantities of active agent and one or more pharmaceutically acceptable excipient(s) through a suitable sieve followed by mixing; b) Granulating the mixture of step a) with an suitable granulation liquid (an aqueous or non-aqueous solvent with optional binder); c) Drying the granulated mass, optionally milling of the dried granules and mixed the sifted granules; d) Lubricating the sifted blend of step c); e) Compressing the lubricated granules into tablets or filling in capsules.

In another embodiment of the invention, there is provided a process for the preparation of a pharmaceutical composition comprising Brivaracetam comprising the steps of: (a) blending a mixture of Brivaracetam and at least one pharmaceutically acceptable excipient; (b) optionally compacting the blended material using roller compactor; (c) optionally milling the compacted material; (d) blend from step (a) or milled material from step (c) was lubricated with a suitable lubricant; (e) lubricated blend or granules from (d) was compressed into tablets with a suitable tooling or filled in capsules.

The composition of the present invention as the term is used herein is a composition of matter including Brivaracetam and other components such as, but not limited to, excipients, like diluent, glidant, surfactant, wetting agent, lubricant, solubilizer, stabilizer, sweetener, flavoring agent, coloring agent and the like.

Embodiments of the present invention also relate to solid oral pharmaceutical compositions of Brivaracetam or its pharmaceutically acceptable salts or derivatives thereof, and at least one or more pharmaceutically acceptable excipient, wherein the composition exhibits at least 85% of drug release within 45 minutes in 900 ml of phosphate buffer, pH 6.4 (Office of Generic Drugs dissolution database) using USP II apparatus (Paddle) at a temperature of 37±0.5° C. and a rotation speed of 50 revolutions per minute (rpm).

Embodiments of the present invention also relate to use of a pharmaceutical composition comprising Brivaracetam or its pharmaceutically acceptable salts or derivatives thereof in the manufacture of a medicament for treating partial-onset seizures, epilepsy, neuropathic pain, essential tremors, Parkinson's disease, dyskinesia, migraine, tremor, essential tremor, bipolar disorders, chronic pain, affective disorders, motor impairment, fibromyalgia, post-herpetic neuralgia, bipolar disorder, schizoaffective disorders, attention disorders, and anxiety disorders.

Various disintegrants include, but are not limited to carboxymethyl cellulose sodium, carboxymethyl cellulose calcium, povidone, crospovidone, polacriilin potassium, sodium starch glycolate, alginic acid, sodium alginate, calcium phosphate tribasic, colloidal silicon dioxide, docusate sodium, guar gum, low substituted hydroxypropyl cellulose (L-HPC), magnesium aluminium silicate, methylcellulose, starch or pre-gelatinized starch and/or combinations thereof. Preferably, the amount of disintegrant is from 0% to about 15.0% w/w.

Pharmaceutically acceptable fillers or diluents include, but are not limited to, microcrystalline cellulose (“MCC”), sodium alginate, silicified MCC (e.g., PROSOLV™), microfine cellulose, lactitol, cellulose acetate, kaolin, lactose, maltose, trehalose, starch, pregelatinized starch, sucrose, mannitol, xylitol, sorbitol, dextrates, dextrin, maltodextrin, compressible sugar, confectioner's sugar, dextrose, polydextrose, simethicone, fructose, calcium carbonate, calcium sulfate, calcium phosphate, dibasic calcium phosphate dihydrate, tribasic calcium phosphate, magnesium carbonate, magnesium oxide and mixtures thereof. Usually, the compositions according to the present invention comprise 0% to about 95% per weight of diluent with respect to the weight of the composition.

Pharmaceutically acceptable binders include, but are not limited to, acacia, guar gum, alginic acid, sodium alginate, dextrin, carbomer, maltodextrin, methylcellulose, ethyl cellulose, hydroxyethyl cellulose, hydroxypropyl cellulose (HPC) (e.g., KLUCEL®), hydroxypropyl methylcellulose (HPMC) (e.g., METHOCEl®), hydroxyethylmethyl cellulose, carboxymethyl cellulose sodium, cottonseed oil, povidone (various grades of KOLLIDON®, PLASDONE®), ceratonia, dextrose, polydextrose, starch, gelatin, pregelatinized starch, hydrogenated vegetable oil type I, maltodextrin, microcrystalline cellulose, polyethylene oxide, polymethacrylates and mixtures thereof. Binder can be present in powder form or as a dispersion or mixture of both. Binder is present in an amount from 0 to about 30%.

Pharmaceutically acceptable lubricants include, but are not limited to, stearic acid, talc, zinc stearate, sucrose stearate, sodium benzoate, hydrogenated vegetable oil type I, calcium stearate, poloxamer, adipic acid, glyceryl palmitostearate, glycerine monostearate, medium-chain triglycerides, sodium stearyl fumarate, glyceryl behenate, sodium lauryl sulphate, sodium stearyl fumarate, magnesium lauryl sulphate, magnesium stearate, polyethylene glycol. Preferably, lubricant is magnesium stearate. The amount of lubricant is from about 0.1% to about 10% w/w. Surfactants or surface-active agents improve wettability of the dosage form and/or enhance its dissolution. Surfactants contemplated in the present invention include, but are not limited to, anionic surfactants, amphoteric surfactants, non-ionic surfactants and macromolecular surfactants. Pharmaceutically acceptable surfactants include, but are limited to both non-ionic and ionic surfactants suitable for use in pharmaceutical dosage forms. Ionic surfactants may include one or more of anionic, cationic or zwitterionic surfactants. Examples include, but are not limited to, sodium lauryl sulfate, monooleate, monolaurate, monopalmitate, monostearate or another ester of polyoxyethylene sorbitan, sodium dioctylsulfosuccinate (DOSS), lecithin, stearyl alcohol, cetostearyl alcohol, cholesterol, polyoxyethylene ricin oil, polyoxyethylene fatty acid glycerides, poloxamer, or any other commercially available co-processed surfactant like SEPITRAP® 80 or SEPITRAP® 4000 and mixtures thereof. Surfactant may constitute from about 0% to about 5% by weight of composition.

Suitable glidants include, but are not limited to, calcium silicate, magnesium silicate, magnesium trisilicate, stearic acid and its derivatives or esters like magnesium stearate, calcium stearate and sodium stearate and the corresponding esters such as sodium stearyl fumarate; talc and colloidal silicon dioxide, tribasic calcium phosphate, starch or mixtures thereof. Preferred glidants are colloidal silicon dioxide and/or talc. Preferably, the amount of glidant is from 0 to about 10.0% w/w.

Other carrier materials (such as anti-adherents, colorants, flavors, sweeteners and preservatives) that are known in the pharmaceutical art may be included in composition of the present invention.

The solid oral tablet dosage form prepared by the process as described in the invention can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high performance liquid chromatography.

In another embodiment, solid oral pharmaceutical composition of the present invention exhibits at least 85% of drug release within 45 minutes in 900 ml of phosphate buffer, pH 6.4 (Office of Generic Drugs dissolution database) using a USP II apparatus (paddle) at a temperature of 37±0.5° C. and a rotation speed of 50 revolutions per minute.

In another embodiment, solid oral pharmaceutical composition of the present invention particularly tablet dosage form of present invention may be packaged in HDPE bottles or blister packs. HDPE bottles may optionally contain desiccants.

As used herein, the term “about” means±approximately 20% of the indicated value, such that “about 10 percent” indicates approximately 08 to 12 percent.

Having described the invention with reference to certain preferred embodiments, other embodiments will become apparent to one skilled in the art from consideration of the specification. The invention is further defined by reference to the following examples describing in detail method for the preparation and testing of Brivaracetam pharmaceutical composition. It will be apparent to those skilled in the art that many modifications, both to materials and methods, may be practiced without departing from the scope of the invention. Following examples are set out to illustrate the invention and do not limit the scope of the present invention.

### EXAMPLES

Brivaracetam tablets were prepared by using quantitative formula as given in Table 1 by preparation method I or II:

TABLE 1

| | Compositions Of Tablet (% w/w) | | | |
|---|---|---|---|---|
| | Exam- | Exam- | Exam- | Exam- |
| Ingredient | ple 1 | ple 2 | ple 3 | ple 4 |
| Brivaracetam | 10 | 12 | 15 | 55 |
| Silicified Microcrystalline | — | 87.5 | 39.25 | 23.5 |
| Cellulose/Microcrystalline | | | | |
| Cellulose | | | | |
| Lactose | 80 | — | 45 | 21.0 |
| Starch | 7.0 | — | — | — |
| L-HPC | 2.0 | — | — | — |
| Colloidal Silicon Dioxide | 0.5 | — | 0.25 | — |
| Magnesium Stearate | 0.5 | 0.5 | 0.5 | 0.5 |

Optionally Film Coating

Preparation Method I (Direct Compression):
i) Brivaracetam and pharmaceutically acceptable excipients were sifted separately through a suitable sieve.
ii) Sifted Brivaracetam was mixed with suitable excipients using a dry mixer.
iii) The mixture obtained at step ii) was optionally milled.
iv) Mixture obtained from step iii)/step ii) was lubricated to obtain the final blend.
v) The blend of step iv) was compressed into tablets using suitable punches.
vi) Optionally the compositions were film coated with dispersion of coating material (Opadry) with approximately 2-3% tablet weight gain.

Preparation Method II (Dry Granulation):
i) Brivaracetam and pharmaceutically acceptable excipients were sifted separately through a suitable sieve.
ii) Sifted Brivaracetam and excipients were compacted using roller compactor.
iii) The granules obtained at step ii) was optionally milled.
iv) Granules obtained from step iii)/step ii) was lubricated to obtain the final blend.
v) The blend of step iv) was compressed into tablets using suitable punches.
vi) Optionally the compositions were film coated with dispersion of coating material (Opadry) with approximately 2-3% tablet weight gain.

The standardized method and equipment for testing dissolution time is provided in Office of Generic Drugs dissolution database. The dissolution profile of tablets dosage form prepared using quantitative composition as given in Example 1, 2, 3 and 4 was measured in 900 ml of Phosphate Buffer, pH 6.4 (Office of Generic Drugs dissolution database) using a USP II apparatus (Paddle) at a temperature of 37±0.5° C. and a rotation speed of 50 revolutions per minute. The dissolution test was conducted to a tablet dosage form as given in Example 1, 2, 3 and 4. The dissolution data is provided in Table 2.

TABLE 2

| Time point | % drug released | | | |
|---|---|---|---|---|
| (minutes) | Example 1 | Example 2 | Example 3 | Example 4 |
| 5 | 106 | 94 | 98 | 92 |
| 10 | 108 | 103 | 99 | 102 |
| 15 | 106 | 106 | 100 | 103 |
| 20 | 107 | 106 | 101 | 103 |
| 30 | 108 | 110 | 101 | 103 |
| 45 | 108 | 108 | 102 | 103 |

Formulations prepared using quantitative composition as given in Example 1, 2, 3 and 4 exhibited more than 85% of drug release within 45 minutes. Further, it was also observed that formulations prepared using quantitative composition as given in Example 1, 2, 3 and 4 were free of surface irregularities like sticking to a punch or die. Also, there was no change in polymorphic form of prepared dosage forms (free from undesirable polymorphic form). The prepared formulation exhibited hardness of at least 4 kiloPascals (kP) as measured by Monsanto hardness tester.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope, and spirit of this invention.

1. An immediate release solid oral pharmaceutical composition comprising brivaracetam or its pharmaceutically acceptable salts, esters, solvates, derivatives, amides, polymorphs, enantiomers, prodrugs, analogues, active metabolites or mixtures thereof and at least one pharmaceutically acceptable excipient, wherein the composition is substantially free of cyclodextrin.

2. The immediate release solid oral pharmaceutical composition of claim 1, wherein the composition is substantially free of binder and/or disintegrant.

3. The immediate release solid oral pharmaceutical composition as claimed in claim 1, wherein the composition comprises less than 0.1% w/w cyclodextrin.

4. The immediate release solid oral aceutical composition as claimed in claim 3, wherein the composition is free of binder.

5. The immediate release solid oral pharmaceutical composition as claimed in claim 3, wherein the composition is free of disintegrant.

6. The immediate release solid oral pharmaceutical composition of claim 1, wherein the composition is substantially free of other polymorphic forms of brivaracetam.

7. An immediate release solid oral pharmaceutical composition comprising:
a) brivaracetam with particle size D90 less than 100 μm; and
b) from about 5.0% to about 90.0% by weight of one or more diluents;
wherein the composition is substantially free of binder and/or disintegrant.

8. An immediate release cyclodextrin free pharmaceutical composition comprising:
(a) from 5% to 80% by weight of brivaracetam;
(b) from 5% to about 95% by weight of one or more diluent;
(c) from 0% to about 30% by weight of one or more binders; and
(d) from 0% to about 15% by weight disintegrant,
wherein, the percentage by weight is relative to the total weight of the composition.

9. The immediate release solid oral pharmaceutical composition of claim 2, wherein the composition is [[brivaracetam]] prepared by a direct compression process.

10. The immediate release solid oral pharmaceutical composition of claim 2, wherein the composition is prepared by a dry granulation process.

11. The immediate release solid oral pharmaceutical composition of claim 1, wherein the composition comprises:
a) brivaracetam with a particle size D90 less than 100 μm; and
b) from about 5.0% to about 90.0% by weight of one or more diluents;
wherein the composition is substantially free of binder and/or disintegrant.

12. The immediate release cyclodextrin free pharmaceutical composition of claim 1, wherein the composition comprises:
(a) from 5% to 80% by weight of brivaracetam;
(b) from 5% to about 95% by weight of one or more diluent;
(c) from 0% to about 30% by weight of one or more binders; and
(d) from 0% to about 15% by weight disintegrant,
wherein, the percentage by weight is relative to the total weight of the composition.
